(19) United States
(12) Patent Application Publication
(10) Pub. No.: US 2023/0321600 A1
(43) Pub. Date: Oct. 12, 2023

(54) AIR PURIFICATION SYSTEM

(71) Applicant: CLEAN AIR ZONE INC., Corona, NY (US)

(72) Inventors: SERGE B. HOYDA, GREAT NECK, NY (US); SRAVANI GANDU, FOREST HILLS, NY (US)

(73) Assignee: CLEAN AIR ZONE INC., Corona, NY (US)

(21) Appl. No.: 17/715,694

(22) Filed: Apr. 7, 2022

(51) Int. Cl. B01D 53/84; A61L 9/14

(52) U.S. Cl. CPC B01D 53/84; A61L 9/14; A61L 2209/111

(57) ABSTRACT

There is an air purification system comprising a housing a fan, at least one water inlet comprising a water inlet valve, at least one biological solution which when combined with water forms a biological agent, at least one pump, wherein there is a computer configured to control the inlet valve, the pump and the fan to control cleaning of the air inside of the housing. Inside the housing is a circulating manifold, at least one tray, at least one air inlet, at least one air outflow, and a plurality of interaction surfaces comprising a plurality of different surfaces slanted at different angles, said plurality of interaction surfaces configured to receive the biological agent.

### BACKGROUND OF THE INVENTION

At least one embodiment relates to an air purification system based on an environmentally safe macrobiotic biological solution. This macrobiotic biological solution when combined with water is called biological agent.

There is an air purification system configured to create an interaction between a biological agent and air flow, wherein the interaction between the biological agent and the airflow cleanses the air. Previously air purification systems would be hindered by lack of interaction between the air flowing through the air purifier and the interaction with the biological agent. Therefore, there is a need to have a plurality of different interaction surfaces configured to receive biological agent so that there is increased interaction with air flow through an air purification system.

### SUMMARY OF THE INVENTION

In at least one embodiment, there is disclosed an air purification system comprising a housing, and a fan disposed inside of or coupled to the housing. There is also at least one water inlet comprising a water inlet valve. The water inlet valve is configured to be connected to a water sources such as a water main. Inside of the housing is at least one biological solution which when combined with water forms a biological agent. The biological agent is configured to be mixed with air so that it cleanses the air of biological material and therefore provides cleaner air from the air purification system. There is at least one pump, wherein the pump is configured to pump biological agent throughout the housing, this biological agent can be pumped through at least one circulating manifold. Once the biological agent is pumped from a lower region in the housing up to a tray, it is spilled out on the tray to then flow down across different interaction surfaces so that this biological agent then extensively interacts with the biological agent to be cleansed. Before, this biological agent interacts with the interaction surfaces it is deposited onto at least one tray. There is also at least one air intake into the housing such as with the grille, and at least one air outlet such as from the fan, drawing air out of the housing.

The plurality of interaction surfaces comprise a plurality of different surfaces slanted at different angles which are configured to receive the biological agent.

There is also at least one computer configured to control the pump, the fan, the water inlet valve, wherein the pump pumps biological agent through the circulating manifold to deliver the biological agent to the tray, wherein the biological agent then flows from the tray to the plurality of interaction surfaces where the biological agent interacts with contaminated air pulled into the housing from the fan such that the air is cleansed by its interaction with the biological agent and then passed to the air outflow through the fan.

### BRIEF DESCRIPTION OF THE DRAWINGS

Other objects and features of the present invention will become apparent from the following detailed description considered in connection with the accompanying drawings which disclose at least one embodiment of the present invention. It should be understood, however, that the drawings are designed for the purpose of illustration only and not as a definition of the limits of the invention.

In the drawings, wherein similar reference characters denote similar elements throughout the several views:

FIG. 1 is a top view of an air purification system which has a fan and a control board;

FIG. 2 is a front view of an air purification system of FIG. 1;

FIG. 3 is a perspective view of the air purification system of FIG. 1;

FIG. 4 is another perspective view of the air purification system of FIG. 1;

FIG. 5 is a bottom perspective view of the air purification system;

FIG. 6A is a side exploded view of the air purification system;

FIG. 6B is a top exploded view of the air purification system;

FIG. 7 is a perspective exploded view of the air purification system;

FIG. 8A is a perspective view of the interior components of the air purification system;

FIG. 8B is a side cross-sectional view of the housing with the pump components disposed therein, having an optional protrusion for catching the cascading biological agent;

FIG. 9 is a schematic diagram of the components controlled by the processor/microprocessor;

FIG. 10 is a close up view of the waterfall system;

FIG. 11 is another close up perspective view of the waterfall system;

FIG. 12A is a front view of the waterfall system;

FIG. 12B is a top view of the waterfall system;

FIG. 13A is a first embodiment of the air purification system incorporated into a HVAC system;

FIG. 13B is a second embodiment of the air purification system incorporated into a HVAC system;

FIG. 14A is another embodiment of the air purification system incorporated into a HVAC system;

FIG. 14B is another embodiment of the air purification system incorporated into a HVAC system;

FIG. 15A is a side cross sectional view of another embodiment of the air purification system incorporated into a HVAC system;

FIG. 15B is a side cross-sectional view of another embodiment of the air purification system incorporated into a HVAC system;

FIG. 16A is a side cross sectional view of another embodiment of the air purification system incorporated into a HVAC system;

FIG. 17A is a side cross-sectional view of another embodiment of the air purification system incorporated into a HVAC system;

FIG. 17B is a side cross-sectional view of another embodiment of the air purification system incorporated into a HVAC system;

FIG. 18A is a top cross-sectional view of another embodiment of the air purification system incorporated into a HVAC system;

FIG. 18B is a side cross-sectional view of another embodiment of the air purification system incorporated into a HVAC system; and

FIG. 19 is a side view of another embodiment of a waterfall system.

### DETAILED DESCRIPTION OF PREFERRED EMBODIMENTS

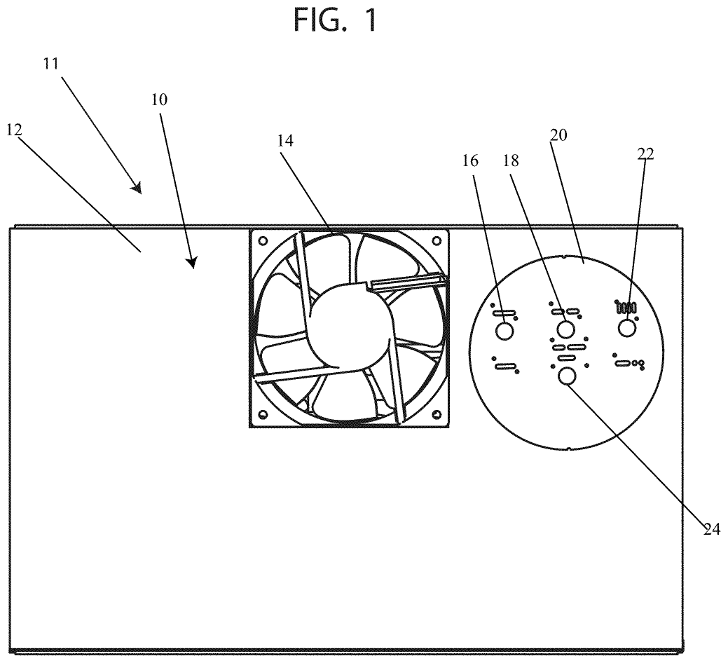

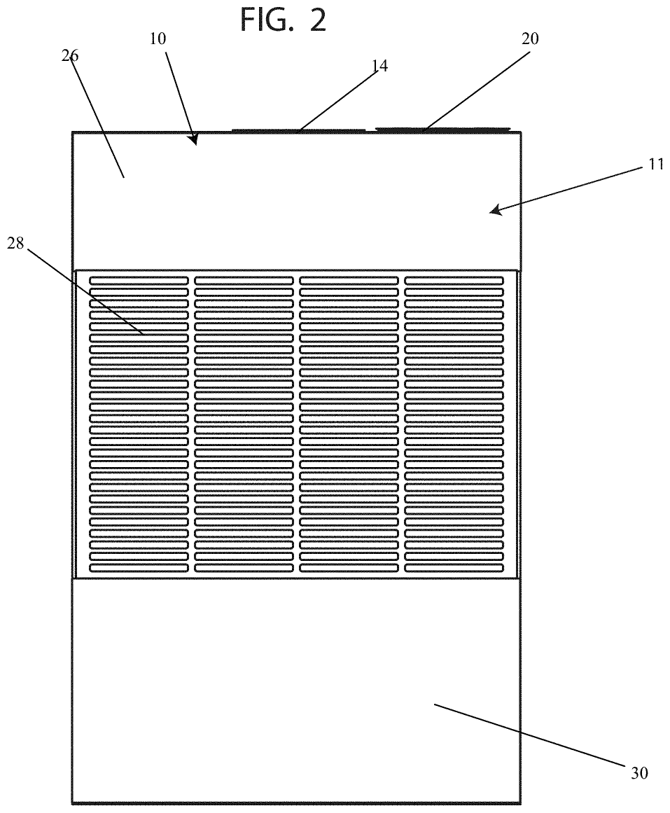

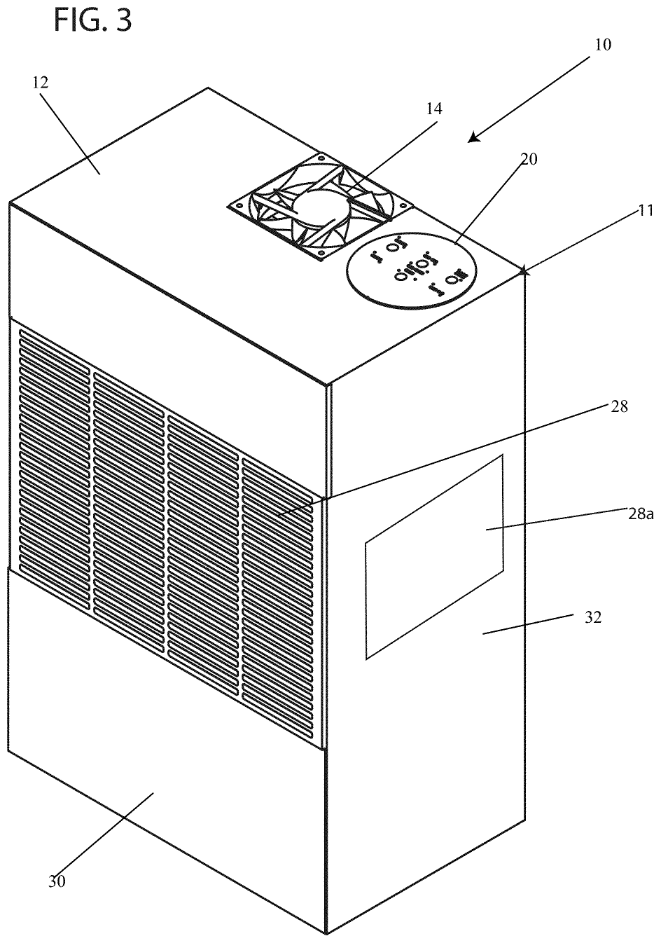

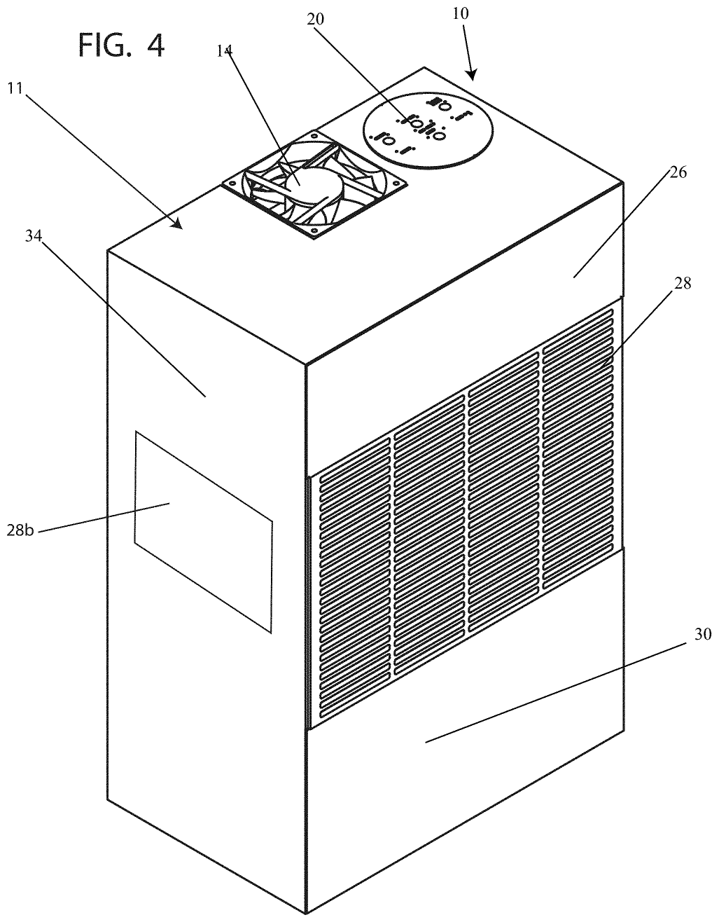

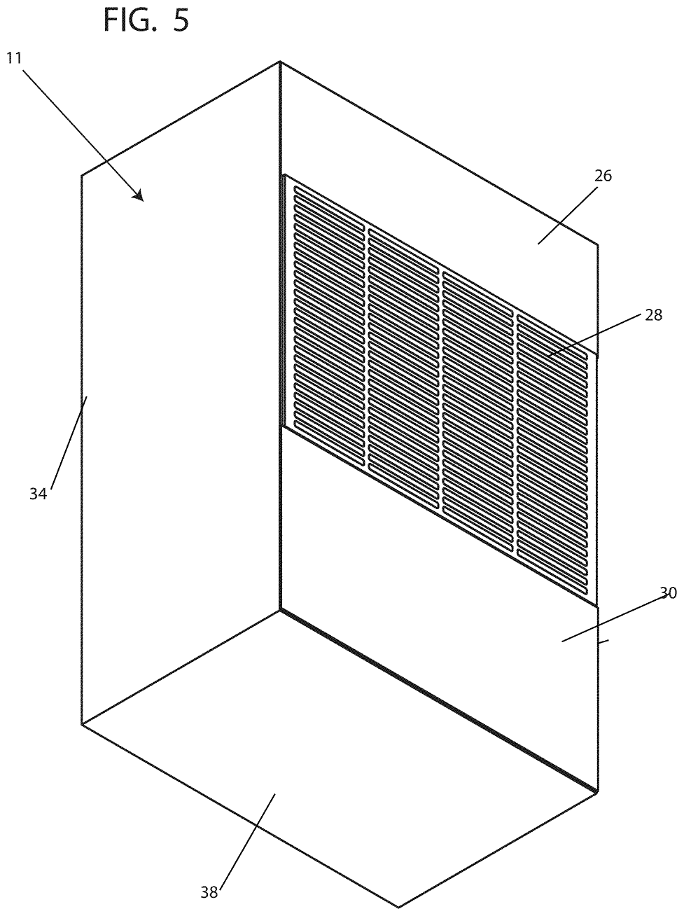

FIGS. 1-5 show the different outer surface views of an air purification system. In at least one embodiment, there is disclosed an air purification system 10 comprising a housing 11, and a fan 14 disposed inside of or coupled to the housing. The housing has a plurality of sides such as sides 12, 26, 30, 32, 34, and 38 (See FIGS. 1-5). There is also at least one water inlet comprising a water inlet valve. The water inlet valve is configured to be connected to a water sources such as a water main. Inside of the housing 11 is at least one biological solution which when combined with water forms a biological agent. The housing forms a container for the biological agent so that it can be pumped to different locations for eventual interaction with air. The biological agent is configured to be mixed with air so that it cleanses the air of biological material and therefore provides cleaner air from the air purification system. For example, while FIG. 2 shows a front view of the system 10 and housing 11, it can also represent a back view of the housing 11 wherein the grill 28 can be positioned on any one or more of the faces of the housing 11. For example, FIG. 3 shows a side view of the housing 11 with an optional grill 28a being placed on side face 32. FIG. 4 shows another optional grill 28b being placed on side face 34. FIG. 5 also shows the bottom face 38.

While multiple different embodiments are shown below, each part of each embodiment is optional in that, the pump, the level sensor, the intake valve, and the fan, any fiber, the number of interaction surfaces are optional in each embodiment etc.

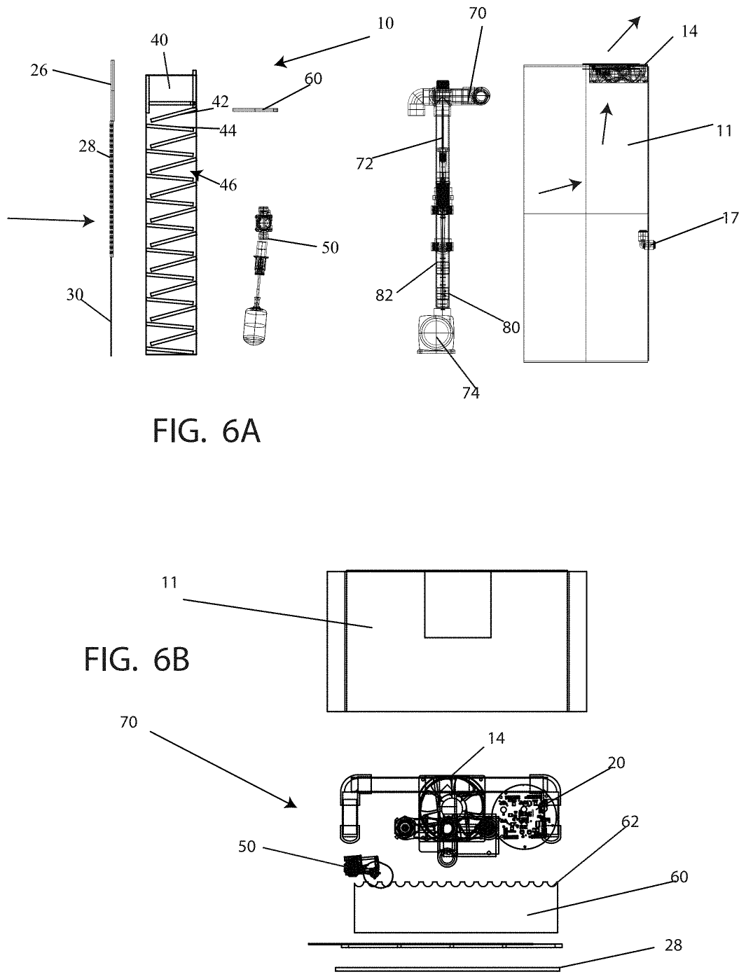

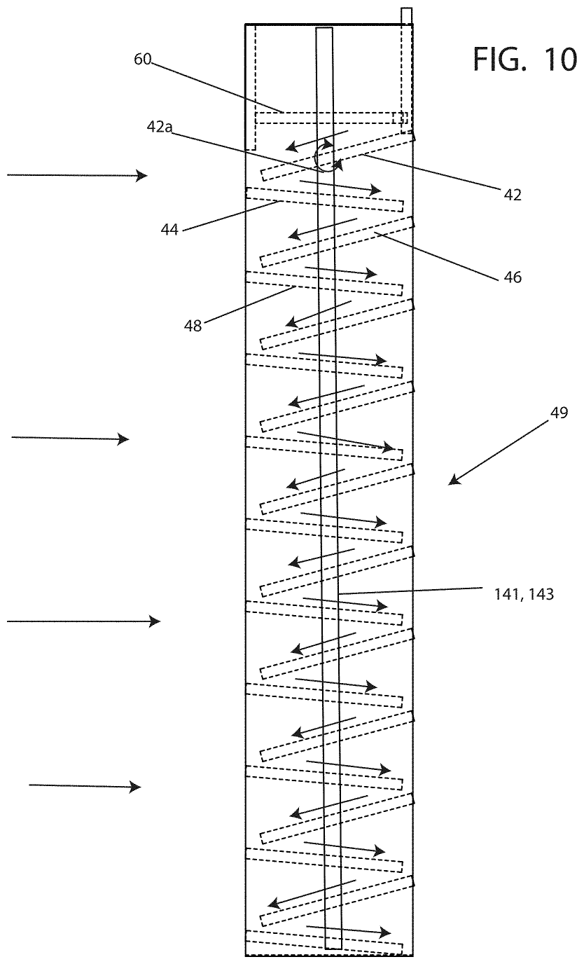

For example, there is shown in FIG. 6A, at least one pump 74, wherein the pump 74 is configured to pump biological agent throughout the housing, this biological agent can be pumped through at least one circulating or pump manifold 72. Once the biological agent is pumped from a lower region in the housing up to a tray 60, it is spilled out on the tray to then flows down across different interaction surfaces 40 comprising a first surface 42, a second surface 44, at least a third surface 46 and a fourth surface 48, there are also additional surfaces 49 below this set of surfaces (see FIG. 10. The first surface 42 is slanted off from horizontal so that this biological agent then extensively interacts with the biological agent to be cleansed. The second surface 44 is slanted in an opposite direction, the third surface 46 is slanted parallel to the first surface and the fourth surface 48 is slanted parallel to the second surface. Before, this biological agent interacts with the interaction surfaces it is dumped onto at least one tray. There is also at least one air inlet and at least one air outflow from the housing wherein the air inlet and the air outflow allow air that is drawn from the fan to flow into the housing and then out of the housing.

The plurality of interaction surfaces comprise a plurality of different surfaces slanted at different angles which are configured to receive the biological agent.

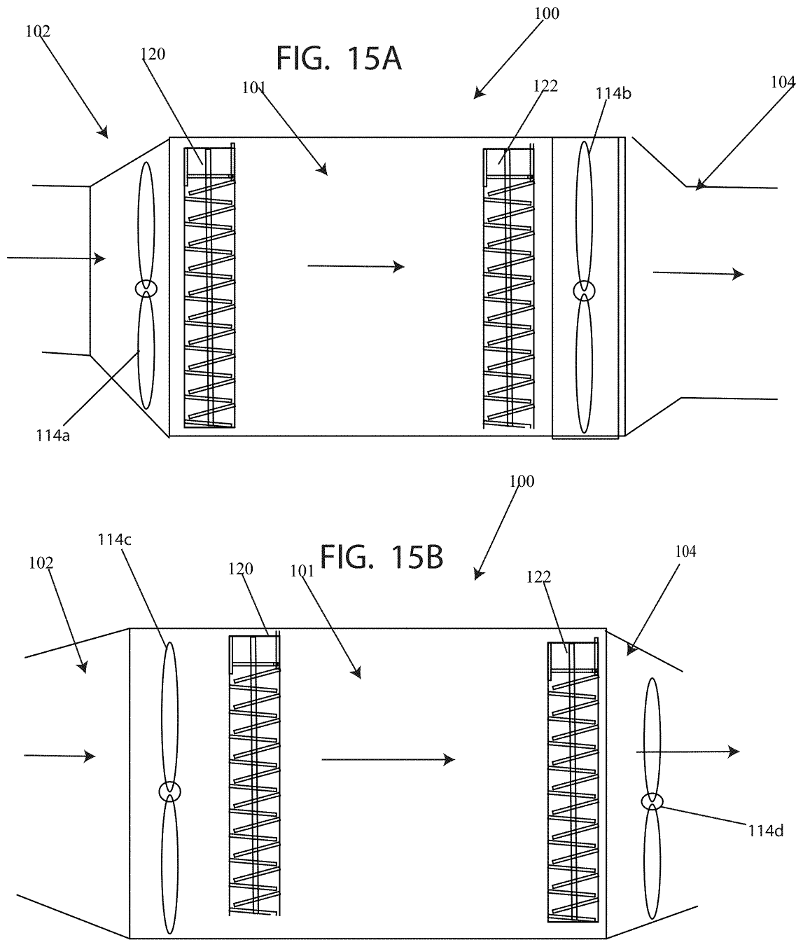

There is also at least one computer 20 configured to control the pump, the fan, the water inlet valve, wherein the pump pumps biological agent through the circulating manifold to deliver the biological agent to the tray, wherein the biological agent then flows from the tray to the plurality of interaction surfaces where the biological agent interacts with air drawn into the housing from the fan such that the contaminated air is cleansed by its interaction with the biological agent and then passed through the Fan out of the housing to the air outside of the housing. The computer can include a processor such as a micro processor which is configured to control all of these components. These components are shown in greater detail in FIG. 15 (see below).

FIG. 6A is a side exploded view of the components. In this view there is shown top front plate 26, grate 28, and bottom front plate 30 forming the front face of the housing. Adjacent to the front face of the housing is the waterfall system 40 which comprises a first set of sloped interaction surfaces 42. This surface is sloped off from horizontal so as to encourage fluid to flow down this interaction surface. Another or second interaction surface 44 is positioned below this first interaction surface 42. This second interaction surface 44 is sloped at an opposite rotation off from horizontal from the first sloped surface. Therefore, in the first sloped interaction surface 42, this surfaces is rotated about a center axis in a clockwise manner so that its front end with respect to the air flow into the housing is below the back end. In contrast, with the second interaction surface 44, the front end of this interaction surface is higher than the back end of the interaction surface with respect to the air flow into the housing. The arrows in this view show the air flow into the housing, and then up towards the fan 14, through the fan and then out of the housing. The first interaction surface 42 can then repeat itself down the waterfall such that these first interaction surfaces are parallel to each other and the second interaction surfaces which repeat each other are also parallel to each other as well. In this way the first interaction surfaces are in an alternating pattern with the second interaction surfaces. The waterfall system includes side walls 46 so that the lateral sides of this waterfall are enclosed.

Disposed above this waterfall system is a tray 60 which is configured to allow the biological agent fluid to flow to the tray first and then flow down into the waterfall system 40. Disposed adjacent to the waterfall system is a level sensor assembly 50. This level sensor assembly has a float which is positioned on a lever and which then, when this float is pushed up due to buoyancy it then causes a water inlet to shut off.

Disposed adjacent to the level sensor assembly 60 is a pump system 70 having a pump 74 pumping biological fluid through a pump manifold 72, having extending arms 72a and 72b, which feed into respective wings 73a and 73b. Each of the arms 72a and 72b have optional holes positioned approximately on a bottom face, for allowing a fluid such as a biological agent to be expelled and cascade down adjacent walls of the housing 11. These holes are shown by way of example by reference numeral 73c wherein these holes can be positioned on a bottom face, or offset from the bottom face but pointing to an adjacent wall such that the fluid such as the biological agent hits the adjacent wall and cascades down this adjacent wall.

The pump manifold 72 has wings 73a and 73b. Pump outlet 76 is configured to spill out over top tray 60. Disposed adjacent to the pump system 70, is another level sensor system 80 having individual level sensors 82. These components are configured to be housing inside of housing 11 having an inflow valve 17.

FIG. 6B is a top view of the system shown in FIG. 6A which shows a housing 11, and a pump system 70. There is shown tray 60 which has notches or cut outs 62 which are semi-circular in pattern. There is also shown grate 28 as well as level sensor assembly 50 and housing 11.

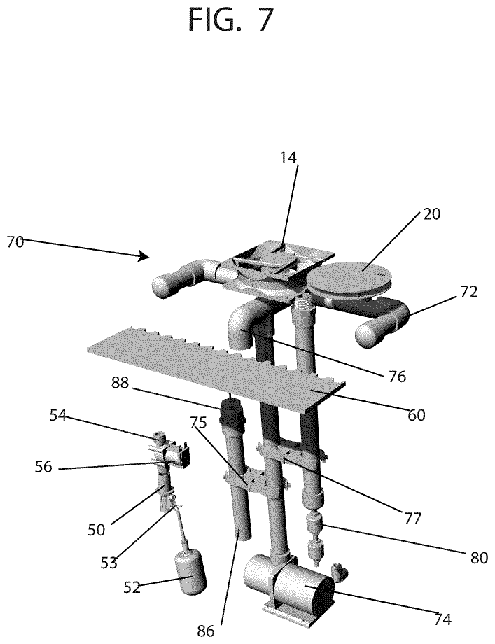

FIG. 7 shows a perspective view of the system that is disposed inside of the housing with the housing and the waterfall system 40 removed. In this view there is shown level sensor assembly 50 having a level sensor float 52, which is coupled to the body section via a hinge 53. The float is rotatable about the body via the hinge 53. At the top of the level sensor assembly 50 is a level sensor controller 56, which selectively opens or closes a valve depending on the position of the position of the float.

As shown in this FIG. 7, the pump system 70 includes the pump outlet 76 which spills onto the tray 60. Disposed below tray 60 is a hydrostatic valve 88 having an inlet pipe 86 extending down to the region where the fluid/biological agent is located. The hydrostatic valve is coupled to the pump manifold system 72 via coupling 75. In addition, another level sensor 80 is coupled to pump manifold system 72 via coupling 77. As shown, disposed above the pump manifold system 72 is fan 14 and controller 20.

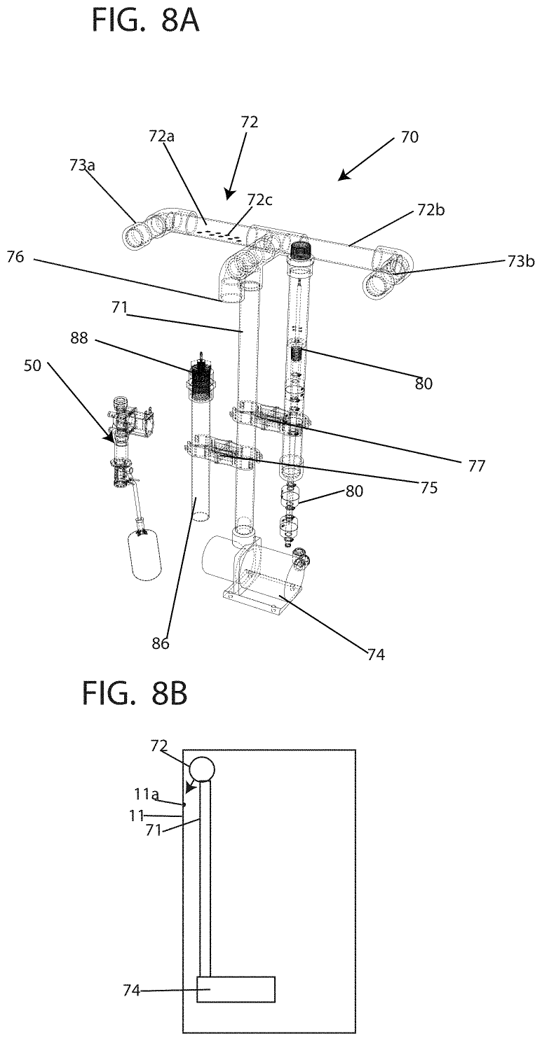

FIG. 8A is a view of the components shown in FIG. 7 wherein these components show the level sensor assembly 50, the hydrostatic valve 88, having pipe 86. There are shown couplings 75 and 77 which are configured to couple the hydrostatic valve 88 to the pump manifold 72 as well as couple the other level sensor 80 to the pump manifold as well. In this view there is shown pump 74 which pumps water up to the top of the housing through a riser pipe 71 of the pump manifold 72. The pump manifold 72 includes wings 73a and 73b which branch off of the manifold to store fluid such as the biological agent that is pumped up from pump 74, before it flows out from pump outlet or downspout 76.

Thus, with this design, biological agent which comprises biological solution mixed with water is circulating in the bottom of the housing 11. Pump 74 receives the biological agent and pumps it through riser in manifold 72. Excess fluid is stored in wings 73a and 73b. The remainder of the fluid biological agent flows out from spout 76 and onto tray 60. This fluid biological agent flows off of tray and onto the waterfall system comprising first set of sloped interactive surfaces, and then down this surface and then down to the second set of sloped interactive surfaces. The biological agent flows to alternating ones of the first set of interactive surfaces and then to the next one until the fluid flows all the way to the bottom of the housing where the remainder of the biological agent fluid is stored. During this time, air flows past these sloped interactive surfaces interacting with each of these surfaces and particularly the biological agent that is covering these surfaces as this waterfall system 40 is in operation.

From time to time additional water may be needed, as measured by either one of the level sensor 50 or the level sensor 80. This causes the controller board 20, to send a signal to open the water inlet valve 17, to allow more water to flow therein to the tank. Both the level sensor assembly 50 and the level sensor assembly 80 can be used to selectively signal when to shut off the water inlet valve 17 so that the housing 11 does not receive too much water. During this time, the hydrostatic valve 88 is configured as a pressure relief valve which relieves pressure from the system so that there is no further pressure inside of the system.

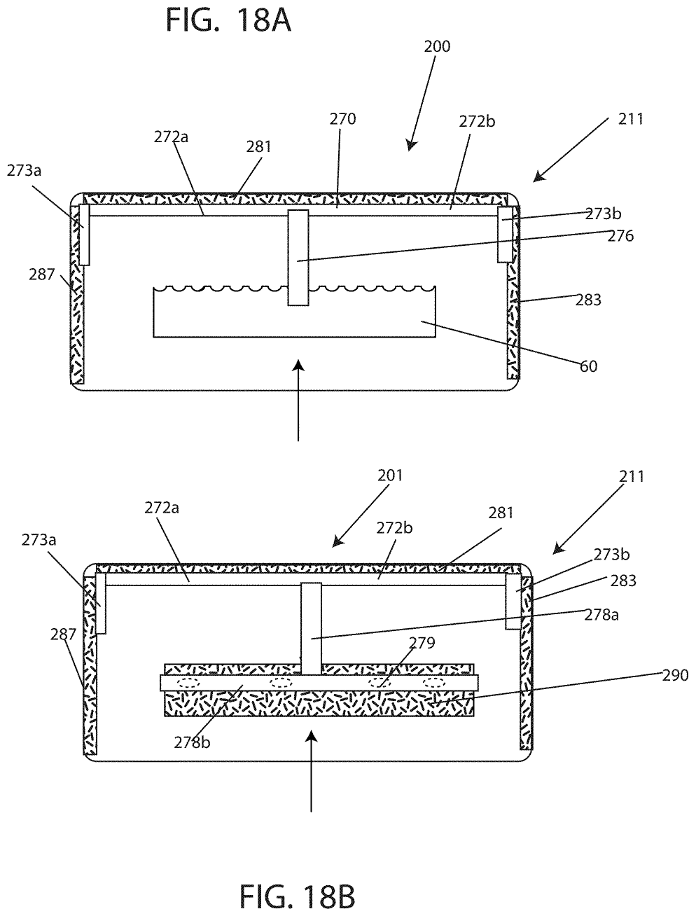

FIG. 8B shows a side cross-sectional view of another embodiment of housing 11. With this embodiment, there is a protrusion 11a, positioned on an inside surface of the back wall of housing 11 towards an upper portion of this back wall. This protrusion can also be placed on the inside surfaces of the side walls as well. This protrusion is shaped as a bull-nose protrusion having a rounded surface to catch the fluid flow down from manifold 72, particularly arms 72a and 72b, as well as wings 73a and 73b having holes 73c (see above) allowing this fluid flow down from this manifold towards the side walls (see arrow pointing to the fluid flow from manifold 72). The bull nose protrusion allows the fluid to catch the protrusion, flow over the protrusion, and then still adhere through surface tension of the fluid against the side walls so that the fluid then flows down these side walls of the housing. The air flow inside of the housing then interacts with these fluid drenched walls to create an air-fluid interaction, thereby cleansing the air inside of the housing before it is expelled by the fan 14. This protrusion and fluid flow from the manifold shown above can also be used with the embodiments shown in FIGS. 18A and 18B below.

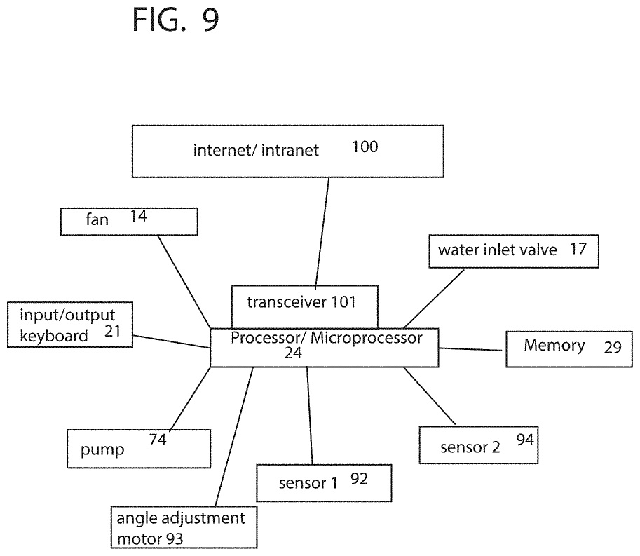

FIG. 9 is a schematic block diagram of the controller system which is used to control the peripheral components. For example, the controller system which is disposed on controller plate 20 includes a processor or microprocessor 24 which is coupled to the input/output keyboard 21. Disposed inside of this controller plate 20 is also a memory 29 as well. The processor/microprocessor 24 is configured to communicate with the internet or intranet 100 via a transceiver 101. Commands can be input into processor/microprocessor 24 via keyboard 21. Processor/microprocessor 24 is configured to control pump 74, fan 14, water inlet valve 17 among the different components. In addition, microprocessor 24 is also configured to read inputs from any one of sensors 92 or 94. These sensors can be any one of hydrostatic valve 88, the level sensor from the level sensor assembly 50, as well as the other level sensors 82 from the other level sensor assembly 80. In addition, other sensors can be connected to this system such as a VOC (volatile air compound sensor) sensor. A particulate senor, a humidity sensor, or any other suitable type of sensor. Microprocessor 24 is also configured to control an angle adjustment motor 93 which is configured to control the angle of each of the interaction surfaces (blades) 42, 44, 46, 48 etc. Each of these surfaces is coupled to a side wall of a waterfall system 40 on an axle 47, which is rotatable about an axis. Each of these axles 47 can be coupled together or separately so that a motor 93 can adjust one or more of these blades/interaction surfaces 42, 44, 46, 48 etc.

FIG. 10 is a side view of the waterfall system 40 which includes the first interactive surface 42, a second interactive surface 44, interactive surface 46 is parallel to first interactive surface and forms part of the first interactive surfaces, while interactive surface 48 is parallel to interactive surface 44. The arrows just above these surfaces show the fluid biological agent flow down these surfaces. The remaining surfaces 49 are shown below these initial surfaces. In addition, there is shown tracks 141 and 143 which on the side of the waterfall system. These tracks 141 and 143 allow this waterfall system to be slid into the housing or slid out of the housing for selective cleaning. The arrows to the left of this waterfall system 40 represent the air flow through or past the waterfall system wherein this air would then interact with the fluid flowing across these interactive surfaces 42, 44, 46, 48 etc. wherein the interaction of the biological agent with the air cleanses the air and draws out the biological impurities in the air and into the biological reagent. This is through the natural biological interaction of the biological agent as well as the electrostatic charge differential between the biological reagent and the air. Each of these interactive/interaction surfaces or blades can be rotated via the angle adjustment motor 93 about an axis so that the angle of each of these interaction surfaces is adjustable. The angle is adjustable to create greater/or lesser interaction/airflow past the interaction surfaces as desired by the user.

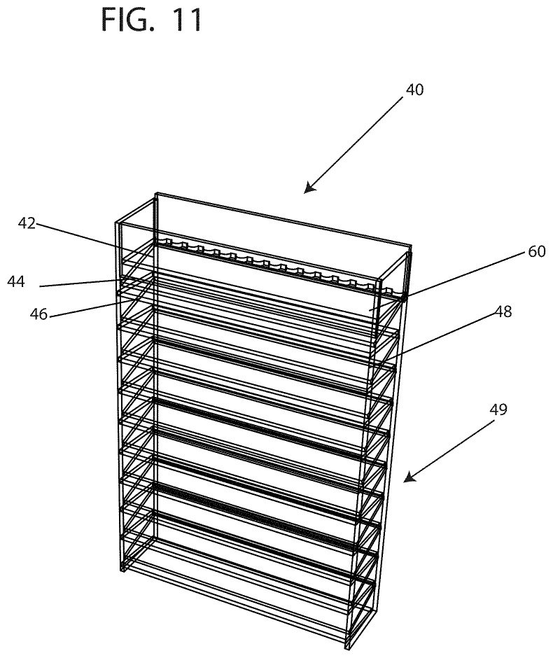

FIG. 11 shows this waterfall system 40 which shows the alternating interactive surfaces 42, 44, 46 and 48, as well as top tray 60. The remaining surfaces 49 are also shown herein.

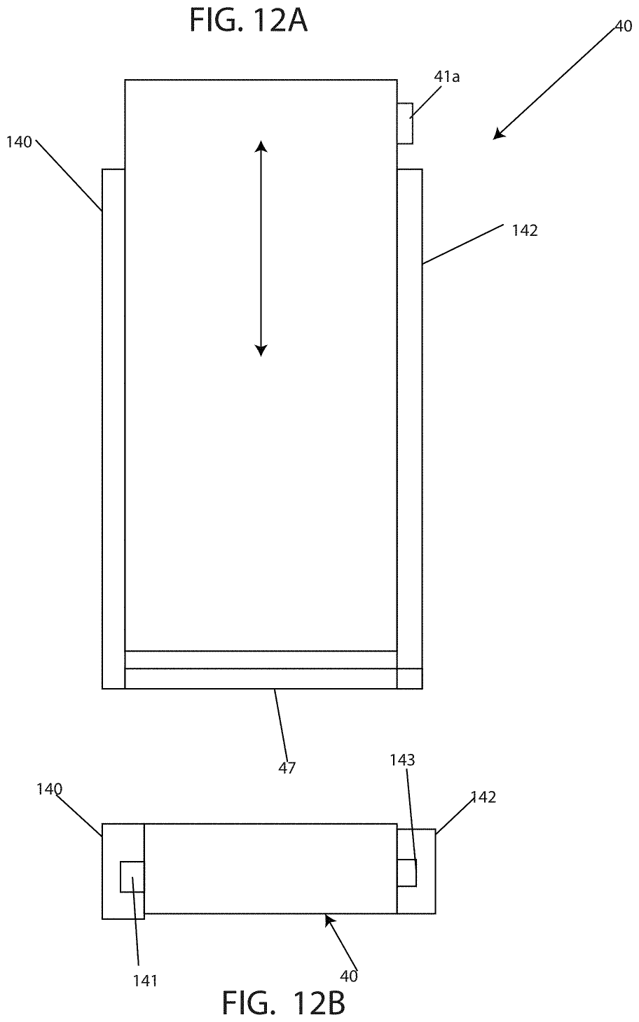

FIG. 12A shows the waterfall system 40 which can be inserted into a track such as track 47 having vertical side tracks 140 and 142 which interact with tracks such as tracks 141, and 143 on a corresponding waterfall system 40 to selectively lock the waterfall system 40 in place so that it stands vertical (See FIG. 12B). In this view, there is also a knob 41a which works similar to angle adjustment motor 93, however this knob 41a is mechanical, and can be activated by the user to rotate the interaction surfaces 42, 44, 46, 48 to change their angle inside the housing 11.

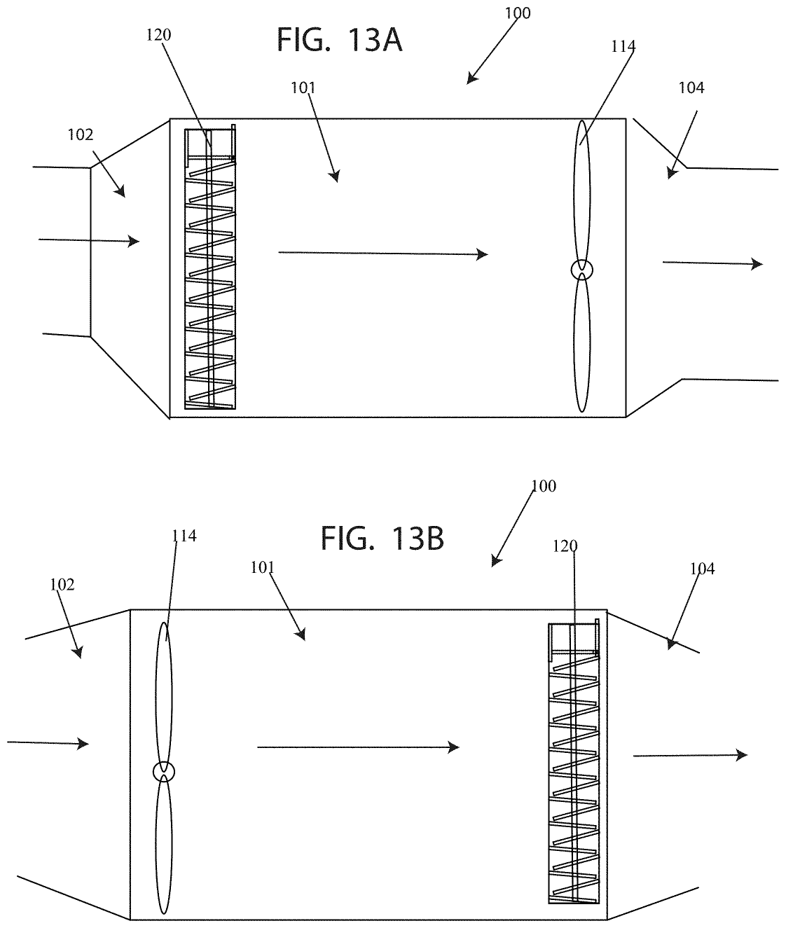

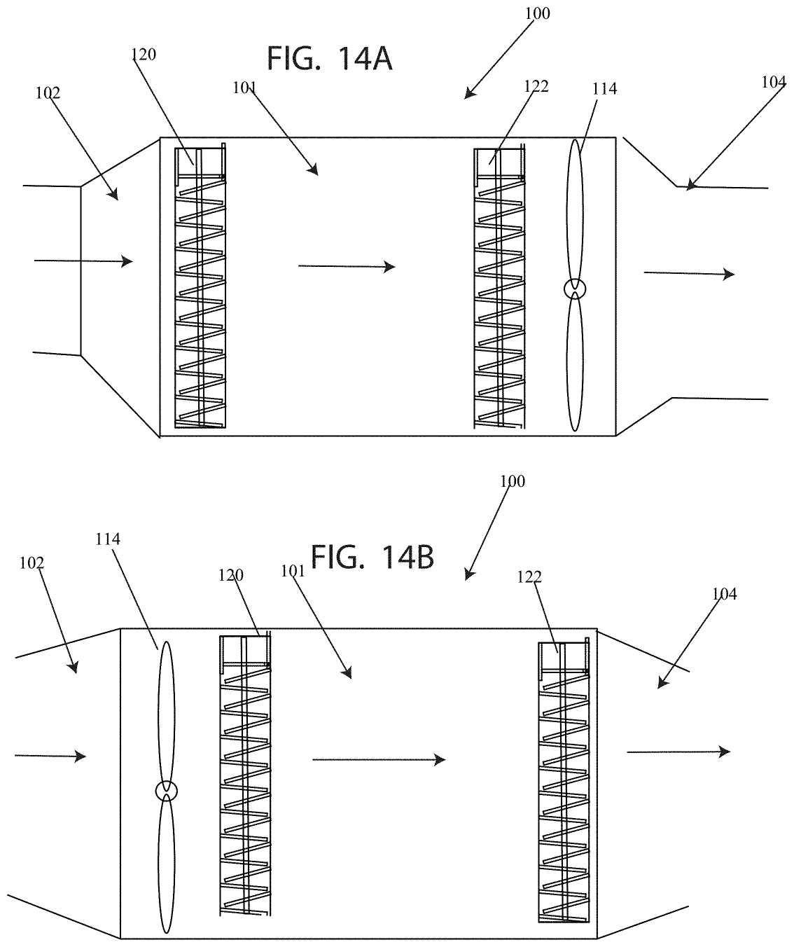

FIG. 13A is a side view of another embodiment wherein this shows a first air handler 100 which has a body section, as well as an inlet 102 and an outlet 104. These parts are also shown in FIGS. 13B, 14A, and 14B. With the design of FIG. 13A, there is shown waterfall system 120 which has a series of interactive surfaces for receiving both the fluid such as the biological agent as well as the air flowing in from the air intake and through to the fan 114. In this embodiment, fan 114 creates a negative pressure inside of housing 101.

Conversely FIG. 13B shows fan 114 positioned before waterfall 120 inside of air handler housing 101. Fan 114 creates a positive pressure inside of housing 101 such that air passes through waterfall system 120 from inlet 102 and through outflow 104.

FIG. 14A shows a cross sectional view of another embodiment, wherein there are two waterfall systems 120 and122 positioned in an air handler, particularly an air handler body 101. With this design the fan 114 creates a negative pressure inside of housing 101 thereby drawing air past each of these waterfall systems so that the water is cleansed by these waterfall systems.

FIG. 14B shows another cross-sectional view of another embodiment, wherein the two waterfall systems 120 and 122 are fed by fan 114 which creates a positive air pressure inside of housing 101. Air then flow from inlet 102 through housing 101 and then outflow 104. In all of the embodiments 13A-14B these waterfall systems are fed by a pump and pump manifold (not shown) but described above in FIG. 7 so that biological reagent is flowing down the interactive surfaces of these waterfalls while air is passing past these interactive surfaces.

FIG. 15A is a side view of another embodiment for use with an air handler 100 having an intake section 102 and an outflow section 104. In this embodiment, there are two different waterfall systems 120 and 122, with the first waterfall system 120 positioned upstream from the second waterfall system 122. There are also two different fans, with fan 114a positioned upstream from waterfall system 120 and fan 114b being positioned downstream from waterfall system 122. While multiple waterfall systems are shown, and multiple fans are shown any number of suitable individual waterfall system or fans can be used. In addition, FIG. 15B shows another version of waterfall systems 120 and 122 positioned inside of air handler 100 with fan 114c being positioned upstream from waterfall system 120 and fan 114 being positioned downstream from waterfall system 122. The arrows inside of each of these air handlers moving from left to right represent the air flow through these air handlers 100.

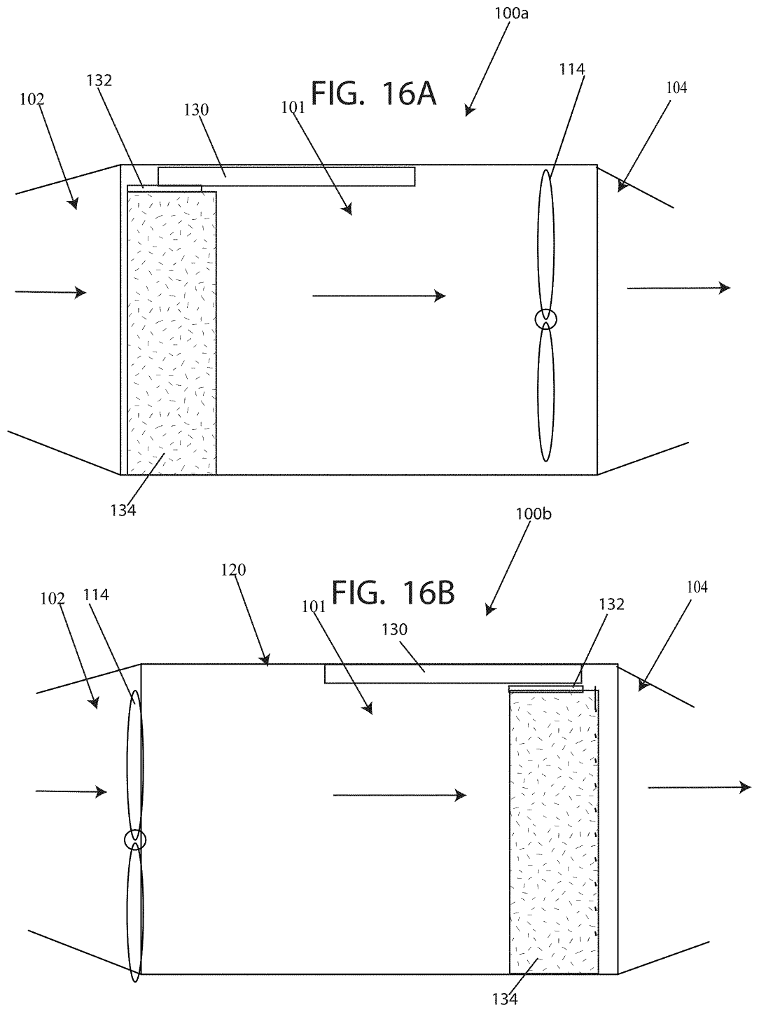

FIG. 16A shows a side view of another air handler system 100a wherein there is an intake 102 and an outflow region 104. There is a fan 114, which promotes air flow from left to right with respect to this orientation through the air handler. There is a fluid manifold 130 which is configured to deliver fluid such as biological agent to a tray 132. Once the fluid hits the tray 132 it spills over to a fiber material 134. This fiber material 134 can be made from any suitable fibrous material such as coconut fiber. The characteristics such as dimensions, density, porosity etc of this fiber material can be adjustable in any suitable manner as is suitable for capturing biological impurities in the air and for cleansing the air. The fiber material becomes filled or impregnated with the biological agent so that it is drenched in this biological agent when air is flowing past it. The numerous interactive surfaces of this fiber material create an interaction zone for the air with the biological agent, thereby causing a cleansing of the air. The biological agent flows down this fiber material 134 to a bottom catch basin (not shown) and then this biological agent is recycled back up to the manifold 130 via a pump (not shown). These features are shown in greater detail in FIG. 7.

FIG. 16B is a side view of another air handling system 100b which shows the fiber material 134 positioned adjacent to the outflow section 104 of the air handler. In this view the fan 114 is positioned adjacent to the inflow section 102 thereby reversing the order of the fiber material 134 and the fan 114 from the embodiment shown above in FIG. 16A. It is understood that multiple sections of fiber material 134 can be used in a single air handler and multiple fans can also be used in a single air handler. In addition, the order of the fans and fiber material can be varied so that the fans can create a positive air pressure against the fiber material 134 or a negative air pressure against the fiber material 134 as well as in the body 101 of the air handler 100.

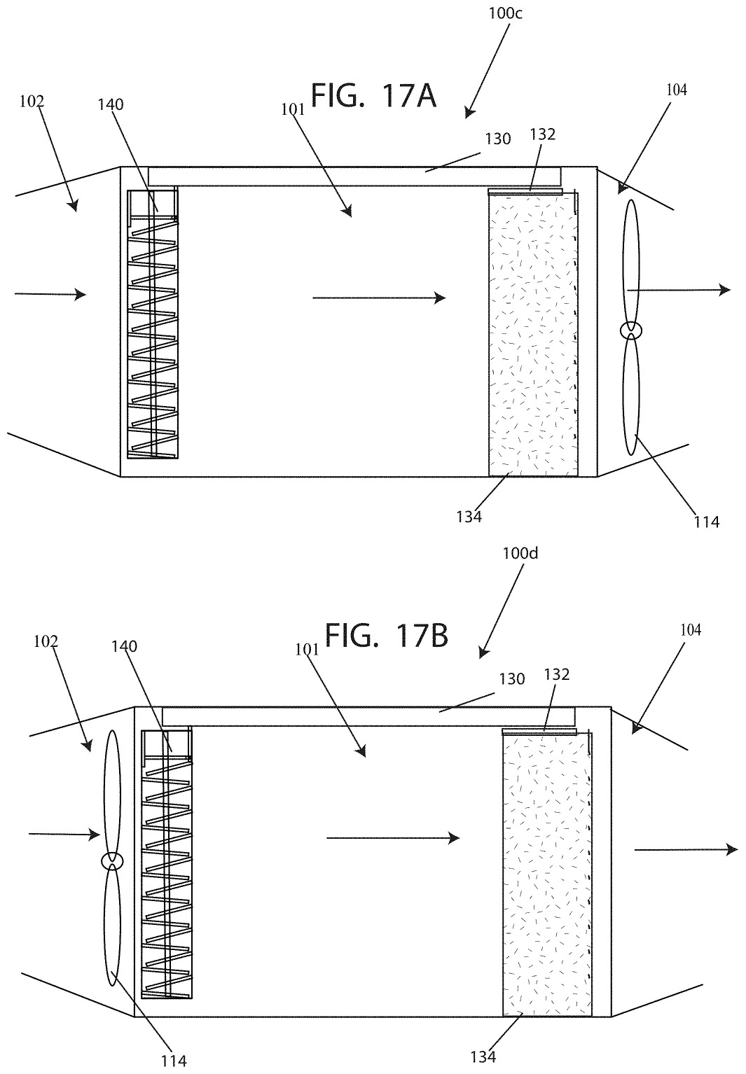

FIG. 17A is another embodiment of an air handler 100c wherein this embodiment there is a waterfall system 140 positioned in a body section 101 of the air handler 100 adjacent to an intake section 102. There is also fiber material 134 positioned adjacent to the outflow section 104 of the air handler body section 101. There is a manifold 130 which feeds fluid to both the waterfall system 140 (see waterfall system 40 above), and to the fiber material 134. Positioned above fiber material 134 is a tray 132 which first catches the fluid which is the biological agent. The fluid then flows down from the manifold 130, past tray 132, and then into fiber material 134. The fluid becomes enmeshed in the fiber material 134 so that when air passes through fiber material 134 it interacts with the biological agent covering this fiber material 134, thereby cleansing the air. Fan 114 is shown in outflow section 104 in FIG. 17A but is shown in inflow section 102 in FIG. 17B.

While a single waterfall system 140 and a single fiber material 134, and a single fan 114 are shown in each of these embodiments of FIGS. 17A and 17B, any suitable number of fiber material 134, waterfalls 140 or fans can be used.

FIG. 18A is a top cross-sectional view another embodiment of the air purification system 200. In this view there is a housing 211 which is similar to housing 11 (see above, particularly FIGS. 1-6B. In this view there is an arrow extending into the body showing the air flow into the housing via a fan (not shown see FIG. 6B). There is a manifold 270 which has arms 272a and 272b which feed into wings 273a and 273b (see arms 72a and 72b and wings 73a and 73b above). There is also a downspout 276 (see downspout 76 above. Each of the arms 272a and 272b as well as the wings 73a and 73b have holes disposed generally on a bottom region to allow for fluid to flow out from these arms and wings and then onto the side walls and/or the fiber 281, 283 and 287 coating the walls. For a view of the fluid flow from this manifold see FIG. 8B above. In addition, the fluid flow out from downspout 276 is such that it flows onto tray 260 and then down an associated waterfall system such as waterfall system 40 shown above in FIG. 6A.

FIG. 18B is another embodiment 201 which shows many of the same components shown above in FIG. 18A such as the fiber 281, 283, and 287 positioned on the respective side walls as well as the manifold arms 272a, 272b, wings 273a and 273b, as well as housing 211. However this design also includes a distribution header 278a and arm 278b which spreads fluid such as the biological agent over the fiber material 290 positioned below this distribution header 278a and arm 278b. In particular, arm 278b has holes 279 positioned along a bottom surface of this arm 278b to allow the biological agent to flow out from arm 278b and onto the fiber material 290. Once the fiber material 290 is flooded with the biological agent, it forms an interactive surface to absorb the contaminated particles or other impurities in the air as the air flows through the housing (see arrow) and then out of the housing 211 via an associated fan (see above showing fan 14 positioned on the top of the housing creating negative pressure inside of the housing 211.

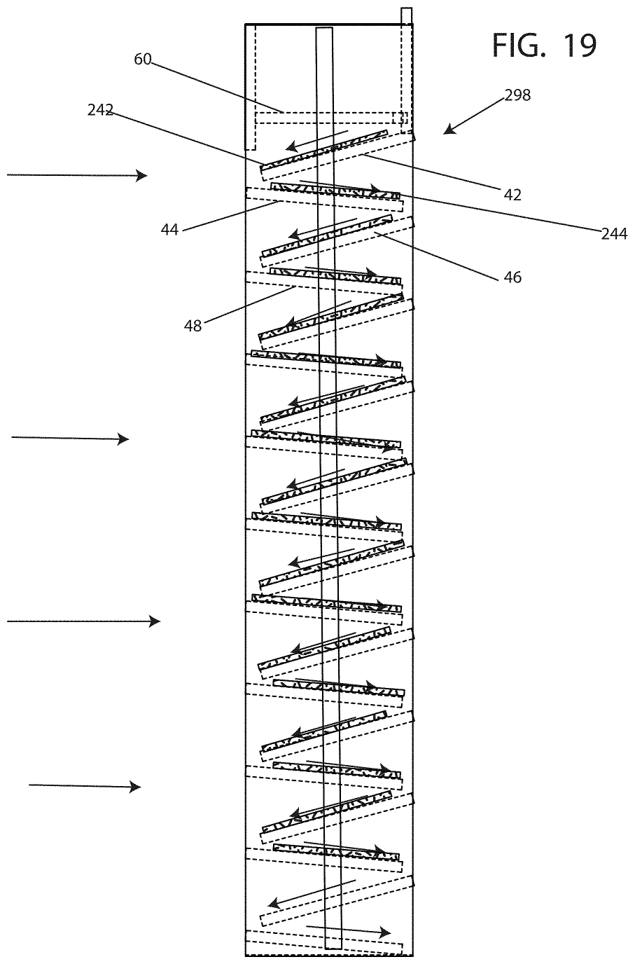

FIG. 19 is another alternative embodiment of the waterfall system 298 such as the waterfall system 40 shown above in FIG. 10. In this embodiment there are optional fiber material sections 242 and 244 for example positioned on interaction surfaces 42 and 44 of the waterfall system. These fiber material sections can be placed on the additional interaction surfaces 46 and 48 or more as needed. As the fluid flows down, it floods the fiber material sections 242 and 244 with biological agent thereby resulting in even more interaction between the biological agent and the contaminated air as the air passes these interaction surfaces having fiber material. Any one of these interactive surfaces can have the fiber material or not as needed. The fiber material is also selectively removable from each and every interactive surface such as surfaces 42, 44 46 or 48. New or additional fiber material may then be placed on these interactive surfaces 42, 44, 46 or 48 as needed.

Accordingly, while at least one embodiment of the present invention have been shown and described, it is to be understood that many changes and modifications may be made thereunto without departing from the spirit and scope of the invention as defined in the appended claims.

What is claimed is:

1. A biological air purification system comprising:
a housing;
a fan;
at least one water inlet comprising a water inlet valve;
at least one biological solution which when combined with water forms a biological agent;
at least one pump;
at least one circulating manifold;
at least one tray;
at least one air inlet;
at least one air outflow;
a plurality of interaction surfaces comprising a plurality of different surfaces slanted at different angles, said plurality of interaction surfaces configured to receive the biological agent;
at least one computer configured to control the pump, the fan, the water inlet valve, wherein the pump pumps biological agent through the circulating manifold to deliver the biological agent to the tray, wherein the biological agent then flows from the tray to the plurality of interaction surfaces where the biological agent interacts with air drawn into the housing from the fan such that the air is cleansed by its interaction with the biological agent and then passed to the through the fan and then out of the housing.

2. The air purification system as in claim 1, wherein the housing is substantially rectangular.

3. The air purification system as in claim 1, wherein the fan is positioned on a top surface of the housing.

4. The air purification system as in claim 1, wherein the computer further comprises a touchpad.

5. The air purification system as in claim 1, wherein further comprising at least one flow level sensor comprising at least one of an electrical or mechanical flow level sensor disposed in the housing and in communication with the computer, wherein when the computer detects that the biological agent is below a predetermined level, the computer opens the water inlet valve to allow water to flow inside of the housing.

6. The air purification system as in claim 5, wherein when the level of biological agent is above a predetermined level inside of the housing, the computer closes the water inlet valve.

7. The air purification system as in claim 1, wherein the computer is configured to selectively speed up the fan or slow down the fan.

8. The air purification system as in claim 1, wherein the computer is configured to selectively speed up the pump or slow down the pump.

9. The air purification system as in claim 1, wherein the computer is configured to selectively shut down the fan and or the pump.

10. The air purification system as in claim 5, further comprising at least one additional level sensor for determining a level of water in the system and then automatically shutting down the water inlet valve when the biological agent reaches a predetermined level inside of the housing.

11. The air purification system as in claim 11, wherein the additional level sensor comprises at least one float coupled to a lever.

12. The air purification system as in claim 1 wherein the housing includes a front grate.

13. The air purification system as in claim 1, wherein the tray includes a plurality of indents.

14. The air purification system as in claim 13, wherein the tray includes a plurality of semi-circular indents.

15. The air purification system as in claim 1, wherein the plurality of interaction surfaces comprise a plurality of different surfaces slanted off from horizontal to at least partially vertical to vertical with slant angles opposite each other so that fluid placed on a first interaction surface flows down the first interaction surface towards a second interaction surface.

16. The air purification system as in claim 15, wherein the plurality of different interaction surfaces comprise at least four different interaction surfaces with a first interaction surface being slanted in a first direction off from horizontal to at least partially vertical a second interaction surface being slanted in a second direction off from horizontal to at least partially vertical, opposite a first surface, wherein the third interaction surface is slanted in an orientation so that it is substantially parallel to the first interaction surface, and wherein the fourth interaction surface is slanted substantially parallel to the second interaction surface.

17. The air purification system as in claim 16, wherein the plurality of different interaction surfaces are disposed in a housing.

18. The air purification system as in claim 17, wherein the housing of the plurality of different interaction surfaces has a protrusion surface, and wherein said housing of the air purification system has tracks configured to receive the protrusion surface such that the housing of the plurality of different interaction surfaces is slidable in and out from the housing of the air purification system.
